(19) United States
(12) Patent Application Publication
(10) Pub. No.: US 2023/0071360 A1
(43) Pub. Date: Mar. 9, 2023

(54) NUCLEIC ACID SEQUENCE DETECTION BY MEASURING FREE MONORIBONUCLEOTIDES GENERATED BY ENDONUCLEASE COLLATERAL CLEAVAGE ACTIVITY

(71) Applicant: Chan Zuckerberg Biohub, Inc., San Francisco, CA (US)

(72) Inventors: Emily D. Crawford, San Francisco, CA (US); Amy M. Lyden, San Francisco, CA (US)

(21) Appl. No.: 17/800,200

(22) PCT Filed: Feb. 23, 2021
(86) PCT No.: PCT/US21/19289

§ 371 Date: Aug. 16, 2022

Related U.S. Application Data
(60) Provisional application No. 62/980,817, filed on Feb. 24, 2020.

(51) Int. Cl. C12Q 1/6816; C12N 9/22

(52) U.S. Cl. CPC C12Q 1/6816; C12N 9/22; C12Q 1/689

(57) ABSTRACT

This disclosure provides methods and materials for determining whether a particular nucleic acid sequence is present in a test sample. Nucleic acid in the sample is treated with a nuclease mixture that generates monoribonucleotides if the target sequence is present. An exemplary nuclease mixture is a CRISPR associated protein that has endonuclease collateral activity (such as Cas12a), in combination with a guide RNA complementary to the target sequence, a reporter oligonucleotide, and an exonuclease. Upon binding of the Cas/gRNA complex to the target sequence in the sample, the endonuclease activity of the Cas protein cleaves the reporter oligonucleotide internally, rendering it suitable for digestion by the exonuclease. Monoribonucleotides that are generated as a consequence are measured by a bioluminescence detection means such as luciferase. The assay method is sufficiently robust, sensitive, and specific for use with a variety of biological test samples, sometimes without amplification.

### REFERENCE TO RELATED APPLICATION

This international (PCT) patent application claims the priority benefit of U.S. provisional patent application 62/980,817 (pending), filed Feb. 24, 2020. In all jurisdictions where permitted, the priority application is hereby incorporated herein in its entirety for all purposes.

### FIELD OF THE INVENTION

This invention relates generally to the detection of particular nucleic acid sequences in a sample and includes assays for the presence of a microorganism or disease condition.

### BACKGROUND

CRISPR technology is revolutionizing functional genomics because it allows precise gene editing. A CRISPR associated protein (Cas), such as Cas9, complexed with guide RNA (gRNA) cleaves cognate target DNA molecules that have a sequence complementary to the gRNA and contain a properly positioned PAM (Protospacer Adjacent Motif). The sequence specificity of CRISPR/Cas system has been adapted for gene editing. See, e.g., Knott, G. and Doudna, J., 2018, CRISPR-Cas guides the future of genetic engineering, Science 361:866-869. In addition, Cas-based biosensing assays have been developed. Reviewed by Li et al., 2019, CRISPR/Cas Systems towards Next- Generation Biosensing, Trends Biotechnol. 37:730-743, 2019; and Zhang et al., 2017, Paired design of dCas9 as a systematic platform for the detection of featured nucleic acid sequences in pathogenic strains, ACS Synth. Biol. 6:211-216.

In addition to site-specific (cis) cleavage of a double-stranded DNA (dsDNA) containing a target sequence, some Cas proteins, when bound to a dsDNA target, also “promiscuously” cleave other nucleic acid molecules that do not contain the target sequence. This non-sequence specific cleavage activity is referred to as Cas “collateral activity” or “trans-cleavage activity.”

Cas collateral activity has been used in development of diagnostic assays, reviewed in Sashital, D., 2018, Pathogen detection in the CRISPR-Cas era: Genome Med 10:32; also see Kocak and Gersbach, 2018, From CRISPR scissors to virus sensors, Nature 557:168-169. For example, the trans-cleavage activity of Cas13a is the basis for a molecular detection platform termed Specific High-Sensitivity Enzymatic Reporter UnLOCKing (SHERLOCK). See Kellner et al., 2019, Nature Protocols 14:2986-3012. The platform has been used to detect specific strains of Zika and Dengue virus, distinguish pathogenic bacteria, genotype human DNA, and identify mutations in cell-free tumor DNA. See Gootenberg et al., 2017, Nucleic acid detection with CRISPR-Cas13a/C2c2, Science 356:438-442. As another example, the trans-cleavage activity of Cas12a has been combined with isothermal amplification to create a sequence detection method termed DNA endonuclease- targeted CRISPR trans reporter (DETECTR). See Chen et al., 2018, Science 360:436-439. DETECTR has been used for specific detection of human papillomavirus in patient samples.

Assay readout in the SHERLOCK and DETECTR platforms is done using a FRET (Fluorescence Resonance Energy Transfer) oligonucleotide probe consisting of a fluorophore and a quencher connected by a short single-stranded DNA oligonucleotide. When the FRET probe and Cas-gRNA are combined in the presence of a sample lacking a target DNA sequence, the Cas collateral activity remains inactive, and the fluorophore moiety in the FRET oligonucleotide probe remains quenched. In the presence of a sample containing the target sequence, the Cas collateral activity is activated, resulting in cleavage of the probe oligonucleotide, physical uncoupling of the fluorophore and quencher, and generation of fluorescence.

The technology provided in this patent disclosure further advances the art of nucleic acid sequence detection.

### SUMMARY OF THE INVENTION

This disclosure provides methods and materials for determining whether a particular target nucleic acid sequence is present in a test sample. Nucleic acid in the sample is treated with a reagent combination that generates nucleotide monophosphate if the target sequence is present. The reagents include an RNA-directed endonuclease (Cas) with collateral activity in combination with a guide RNA complementary to the target nucleic acid sequence, a reporter oligonucleotide with blocked termini, and an exonuclease. Upon binding of the Cas/gRNA complex to the target sequence in the sample, if present, the collateral activity of the Cas protein cleaves the reporter oligonucleotide internally, rendering it a substrate for digestion by the exonuclease. Nucleotide monomers (NMPs), e.g., adenosine monophosphate monomers (AMP), generated as a consequence of exonuclease activity are measured. Typically, NMPs (e.g., AMP) is converted to adenosine triphosphate ATP and ATP is detected by a bioluminescence detection means such as luciferase reporter assay. The assay method described herein is robust, sensitive, and specific and may be used for a variety of biological test samples.

Embodiments of the invention are featured in the description that follows, the drawings, and in the appended claims.

### DRAWINGS

FIG. 1A depicts the general principle of some of the assay methods provided in this disclosure. Upon activation of a CRISPR associated protein in the presence of its target DNA sequence, collateral cleavage activity cuts a detector probe at one or more internal sites, rendering them amenable to degradation by an exonuclease into single nucleotides. The detector probe comprises an oligonucleotide comprising nucleotides (e.g., adenosine monophosphate) and has blocked ends prior to cleavage. The released nucleotides are converted to ATP, which in turn is detected by bioluminescence. In the absence of the target DNA sequence, the Cas collateral activity remains inactive, the probe remains blocked, and no adenosine nucleotides are produced.

FIG. 1B depicts a particular implementation of the method. In Step 1, Cas12a is complexed with a guide RNA targeting a sequence of interest. A reporter oligonucleotide present in the reaction mixture is a blocked polyadenosine oligonucleotide. In Step 2, RNAse T (also called Exonuclease T or Exo T) degrades any Cas12a-cleaved detector fragments from the exposed 3′ end. In Step 3, AMP monomers are detected by a luciferase reporter system.

FIG. 2 shows data from an assay used to detect the fbiC gene from Mycobacterium tuberculosis. The assay detects the target DNA at a concentration of 25 nM.

FIG. 3 shows data from an assay repeated with varying concentrations of target DNA. A concentration of 500 pM of the fbiC gene is detectable.

### DETAILED DESCRIPTION

### I. Overview and Advantages

This invention provides an improved system for detection of predetermined nucleic acid sequences (“Target DNA”) in a sample containing nucleic acid. Detection of the target DNA is coupled to a luminescent readout. The assay methods described herein may generally be referred to using the mark NANCI™, without limiting practice of the invention. NANCI is an acronym for exoNuclease-Assisted endoNuclease Cleavage Indicator™. The system can be implemented as a homogenous assay, meaning that a signal is produced after mixing of sample and reagents, without requiring a separation step. In one aspect the invention provides an assay method for determining whether a nucleic acid sample contains a target sequence by combining the sample with a nuclease mixture that generates monoribonucleotides from a reporter oligonucleotide if the nucleic acid in the sample contains the target sequence; quantitating monoribonucleotides generated; and determining the quantity of monoribonucleotides generated whether the nucleic acid sample contains a target sequence.

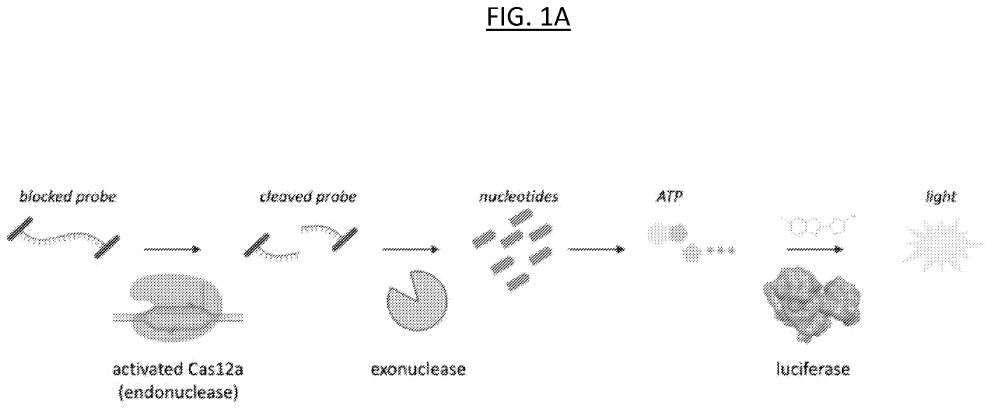

FIG. 1A depicts the general principle of one embodiment of a NANCI assay. It will be understood that there are numerous variations and substitutions that can be used in the assay. In one approach, the assay shown in FIG. 1A employs a synthetic RNA molecule that is blocked to prevent exonuclease degradation. Upon binding of a CRISPR associated protein (e.g., Cas12a)-gRNA to its target DNA, the Cas trans-cleavage activity is activated and the enzyme cleaves the probe at one or more internal sites, producing unblocked fragments amenable to exonuclease degradation. An exonuclease is present or is introduced, and the oligonucleotide probe is degraded into monoribonucleotides (AMP, CMP, GMP and/or UMP, or analogs thereof). Nucleotides (e.g., adenosine monophosphate) generated by the exonuclease activity are converted, directly or indirectly, to ATP. In one approach, the ATP is in turn consumed in a luciferase reaction that generates light which can be read by a luminometer or similar device, or in some cases by the human eye. In the absence of target DNA, the Cas remains inactive, the probe remains blocked, and no monoribonucleotides are available for the luciferase reaction.

It will be recognized that various combinations of Cas proteins, nucleic acid reporter probes, blocking moieties, and exonucleases may be used in the assays, and that the choice of each component may affect the choices of other components. For example, a Cas protein with endoRNAse collateral activity may use reporter probes that are fully or partly RNA (i.e., rNMP polymers) while a Cas protein with endoDNAse collateral activity may require a mixed or chimeric reporter probe with a DNA portion that is a substrate for the endoDNAse activity and an RNA portion that is a substrate for an exoRNAse and may be digested to produce rNMPs (e.g., rAMP). Exonuclease(s) may be selected based on the composition of the reporter probes (for example, an exoRNAse may be used to digest an RNA, while an RNA/DNA chimera may need require an exonuclease, such as RNAse T, that digests both RNA and DNA polymers, or a combination of exonucleases that provide this dual activity. See Zuo, Y. and Deutscher, M., 2002, “The Physiological Role of RNase T Can Be Explained by Its Unusual Substrate Specificity” J Biol. Chem. 227: 29654-29661. It will be recognized that selection of appropriate blocking moiety(s) will depend on both the composition of the reporter probe and the specificity of the exonuclease(s). For example, the presence of 3′ terminal cytidine residues blocks digestion by RNase T. See Viswanathan et al., 1998, “Identification of a Potent DNase Activity Associated with RNase T of Escherichia coli” J. Biol. Chem. 273:35126-35131.

Assay methods of the invention described here are more sensitive than prior art methods, lack interference from background autofluorescence, and have the advantage that signal can be detected in the absence of an excitation source, making use in low-resource settings more feasible. Possible applications include detection of pathogens, detection of antimicrobial resistance sequences, SNP analysis and mutation detection.

### II. Cas Proteins with endoRNAse Collateral Activity

As noted above, some Cas proteins have a Cas endonuclease collateral activity. Substrate(s) for the collateral activity of a specific Cas protein can be single-stranded (ss) DNA (e.g., substrate for Cas 12a and Cas 14), single-stranded RNA, (e.g., substrate for Cas 13), synthetic analogs of DNA or RNA, chimeric RNA/DNA oligonucleotides. These nucleic acid substrates can be polymers of mono(deoxy)ribose nucleotide monomers. In some approaches the nucleic acid substrates are RNAs. In some embodiments the nucleic acid substrates are mixed polymers in which some monomers are rNMP and some monomers are dNMP. An example of a chimeric nucleic acid substrate, for illustration, is “5′-rArArArAdTdTdTdG-3′ [SEQ ID NO:1]. Nucleic acid substrates (including substrates comprising terminal blocking groups) may be referred to as “reporter oligonucleotides” or “nucleic acid reporter probes” or “reporter probes”

This disclosure provides an assay method for determining whether a nucleic acid sample contains a polynucleotide (sometimes referred to as a “target polynucleotide”) with a specified target sequence. The target polynucleotide may be RNA or DNA. An example of a target sequence recognized by an RNA-Cas protein complex is a DNA sequence characteristic of a specific pathogen. In some cases, recognition and binding of a target polynucleotide by a RNA-Cas protein complex requires a properly positioned protospacer or “PAM” sequence on the target polynucleotide. In this case, the combination of the PAM sequence and the sequence recognized by gRNA can be referred to together as the target sequence.

As discussed above, the target sequence is recognized by ribonucleoprotein complex containing the Cas protein and one or more associated RNAs (i.e., the “guide RNA”) with sequence complementary to target sequence. For example, the Cas protein-gRNA complex can comprise a crRNA (CRISPR RNA), a 17-20 nucleotide sequence complementary to the target DNA, and a tracr RNA, which serves as a binding scaffold for the Cas nuclease. Alternatively, the complex can comprise Cas protein and a single guide RNA (sgRNA), in which both crRNA and tracrRNA are fused in a single molecule. These RNAs (and any RNAs that form a complex with a Cas and confer target specificity to the target) are referred to in this disclosure as “gRNA.” Guide RNAs may be designed using art known means to recognize a desired target sequence, and combinations of guide RNAs and endonucleases may be designed for panels or multiplexing applications.

Cas proteins may have collateral RNA endonuclease activity (also called endoribonuclease activity). That is, without intending to be bound by a particular mechanism, upon recognition or binding of a target sequence in a DNA (ssDNA or dsDNA) target polynucleotide or an RNA (ssRNA or dsRNA) target polynucleotide, the RNA endonuclease activity is activated as a collateral activity. Alternatively, Cas proteins may have collateral DNA endonuclease activity (also called endodeoxyribonuclease activity). When a Cas protein with collateral endodeoxyribonuclease activity is used, the nucleic acid reporter probe will be selected or designed to be susceptible to cleavage by an endodeoxyribonuclease. For example, a chimeric molecule comprising a DNA portion as a substrate for cleavage and an RNA portion (as a source of nucleotide monomers) may be used. In some cases, the Cas protein may have both RNA endonuclease activity and DNA endonuclease activity.

Exemplary Cas proteins with collateral RNA endonuclease or DNA endonuclease activity are listed in Table 1. It is contemplated that homologs, orthologs, paralogs, analogs and engineered variants of the listed proteins may be used. It will be within the capability of a person of ordinary skill in the art who is guided by this disclosure to identify Cas protein(s) with endoribonuclease activity and suitable for the NANCI assay.

TABLE 1
Exemplary Cas Proteins With Collateral RNA Endonuclease Activity

| | Sequence | | |
|---|---|---|---|
| | Recognition and | | |
| | Binding activity of | Collateral activity | |
| | Cas/gRNA complex | substrate | Representative Reference |
| Cas12¹ | dsDNA | ssDNA-RNA | Chen et al., 2018, Science 360:436-439 |
| | | chimera, RNA, | |
| | | dsDNA² | |
| Cas13³ | ssRNA | ssRNA | Gootenberg et al., 2017, Science |
| | | | 356:438-442 |
| Cas14 | ssDNA | ssDNA-RNA | Harrington et al., 2018, Science |
| | | chimera | 362:839-842 |

1. Including Cas12 a, b, c, g, h, i
2. Trans-cleavage activity of Cas12a also affects RNA and dsDNA, though less efficiently than ssDNA
3. Including Cas13a, Cas13b, Cas13c, Cas13d, Cas13a6

It will be recognized that Cas proteins with RNA endonuclease collateral activity will cleave RNA or an RNA-DNA chimera, and Cas proteins with DNA endonuclease collateral activity will cleave an RNA-DNA chimera. A person of ordinary skill in the art guided by this disclosure will be able to match Cas proteins and reporter oligonucleotides suitable for the NANCI assay. In this context, a DNA-RNA chimera refers to a single-stranded polynucleotide comprising both deoxyribonucleotides and ribonucleotides. For example, the oligonucleotide 5′-(rA)ₙ(dG)ₓ-3′ would be cleaved by an endonuclease with a ssDNA collateral activity, and could be digested by a 3′ exonuclease that recognizes both ssDNA and RNA as a substrate.

### III. Choice of Reporter Probes and Exonucleases

The chemical nature of the reporter oligonucleotide used to detect Cas protein collateral activity will depend on the specificity requirements of the protein (Table 1). To the extent such variations are tolerated by the collateral activity of the Cas protein being used, the reporter oligonucleotide may use non-naturally occurring nucleosides, nucleoside analogs, and alternative background chemistries. These are taught, for example, in the book Modified Nucleic Acids (Nucleic Acids and Molecular Biology), K. Nakatani et al. eds., Springer 2016.

Certain advantages of the assay method of this disclosure are achieved by using an exonuclease to liberate monoribonucleotides from the reporter oligonucleotide after it is first cleaved by collateral activity of the Cas protein. The exonuclease chosen for a particular implementation of the assay depends on the chemical make-up of the reporter oligonucleotide, which in turn depends on the collateral activity of the Cas protein that is used for detecting the target nucleic acid sequence. For example, where the Cas protein is Cas13 and the reporter oligonucleotide is a single stranded RNA, the exonuclease is generally an RNA exonuclease.

Within these constraints, a variety of exonucleases, and combinations of exonucleases, can be used. The exonuclease may have 5′-3′ exonuclease activity, 3′-5′ exonuclease activity, or both 5→3′ and 3→5′ exonuclease activity, or may be a combination of multiple exonucleases with one or both activities. See R. F. dos Santos et al., 2018, “Major 3′-5′ Exoribonucleases . . . ” Mol. Biol. Translational Sci. 2018; Y. Zho et al., 2018, “Exoribonuclease superfamilies,” Nucleic Acids Res. 29:1017-1026. It will be recognized that digestion by some exonucleases may produce other products (e.g., dinucleotides) in addition to monoribonucleotides. The exonuclease may be specific for termini of RNA, of DNA, or of both RNA and DNA as substrates. In some cases, an exonuclease specific for RNA, and an exonuclease specific for DNA may be used in combination. Suitable exonucleases will generate free monoribonucleotides (or dimers, etc., that are converted to mononucleotides by another reaction reagent).

Selection of a particular exonuclease places certain requirements on the nucleic acid sequence of the reporter probe being used, so that it will be a suitable substrate for the processive activity of the exonuclease to generate monoribonucleotides once cleaved by the collateral activity of the Cas protein. For example, RNAse T (also known as Exonuclease T or Exo T) is a single-stranded RNA or DNA specific nuclease that requires a free 3′ terminus and removes nucleotides in the 3′→5′ direction. Exo T has minimal activity if the free 3′ terminus is oligocytidine. Thus, 3′-terminal oligocytidine may be used to block digestion by Exo T.

In one approach a suitable reporter oligonucleotide for an assay with RNAse T is rich in rA (e.g., polyadenosine). An advantage of rA-rich reporters is that a large number of AMP molecules may be generated by the exonuclease, and free AMP is readily converted to ATP which may be used in many luciferase-based assays. See, e.g., Resnick, M. and Zehnder, A., 2000, “In Vitro ATP Regeneration from Polyphosphate and AMP by Polyphosphate:AMP Phosphotransferase and Adenylate Kinase from Acinetobacter johnsonii210A” Appl Environ Microbiol. 66(5): 2045-2051; Mondal et al., 2017, “Utility of Adenosine Monophosphate Detection System for Monitoring the Activities of Diverse Enzyme Reactions,” ASSAY and Drug Development Technologies, November 2017.330-341; Promega, 2017, “Technical Manual UMP/CMP-Glo™ Glycosyltransferase Assay” (available at www at.promega.com/imedia/files/resources/protocols/technical-manuals/500/ump_cmp-glo-glycosyltransferase-assay.pdf? la=en); and U.S. Pat. Nos. 6,602,677, 7,241,584, 8,030,017 and 8,822,170, each of which is incorporated by reference herein. In some embodiments the reporter oligonucleotide has a length of at least 20, at least 30, at least 40, at least 50, at least 60, at least 70, at least 80, at least 90, at least 100, at least 110, or at least 120 nucleotides and the reporter is at least 50% adenosine, at least 60%, at least 70%, at least 80%, at least 90%, or at least 95% adenosine. In some embodiments the reporter oligonucleotide has at least one, two, three or more stretches of at least 5, 10, 20, 60, 100 consecutive adenosines each.

Typically, the reporter oligonucleotide is blocked at the 5′ or 3′ terminus. A reporter oligonucleotide in a reaction is “blocked” at one or both termini if the reporter oligonucleotide it has properties that prevent the exonuclease used in the assay from processively digesting from the terminus. For example, the reporter probe of SEQ ID NO:2 is blocked at the 3′ terminus for Exo T, for reasons discussed above. rA indicates an adenosine ribonucleotide, rC indicates a cytidine ribonucleotide, and n is 17 to 200.

[SEQ ID NO: 2]
5′-(rA)ₙrCrCrC-3′

Likewise, the reporter probe of SEQ ID NO:3 is blocked at the 5′-end for a 5′-3′ exonuclease that is not active on a DNA substrate.

[SEQ ID NO: 3]
5′-dAdAdArArArArArArArArArArArArArArArArArA-3′

The nature or properties of the reporter oligonucleotide will determine or influence the selection of the nature of the terminal blocking moiety that prevents exonuclease digestion in the absence of collateral activity.

Either or both termini of the reporter oligonucleotide may be blocked. For example, if the nucleic acid substrate is ssRNA and the exonuclease is RNAse T, it is usual to block at least the 3′ terminus, because RNAse T has 3′→5′ exonuclease activity. Blocking the 5′ terminus is optional because RNAse T does not have 5′→3′ exonuclease activity). In contrast, if the nucleic acid substrate is ssRNA and the exonuclease is Bacillus subtilis RNase J1, a 5′→3′ exodeoxyribonuclease (Mathy et al., 2007, “5′-to-3′ exoribonuclease activity in bacteria: role of RNase J1 in rRNA maturation and 5′ stability of mRNA” Cell 129:681-92) it is usual to block the 5′ terminus, but blocking the 3′ terminus is optional. Likewise, the nature of the terminal blocking moiety to prevent exonuclease digestion of the intact nucleic acid substrate can vary depending on the substrate and exonuclease.

When used with RNAse T, a reporter oligonucleotide of polyadenosine residues may be blocked for purposes of use with RNAse T using one or more cytidine residues at the 3′ end. Alternative blocking groups may include phosphorylation at either end, a 3′ dideoxinucleotide, or phosphorothioated bonds at either end, among other things. Also suitable depending on the context is structural blocking, such as formation of hairpin structures, branched structures. See A. L. Steckelberg et al., 2018. “A folded viral noncoding RNA blocks host cell exoribonucleases” Proc. Natl. Acad. Sci. USA 15: 6404-6409.

As described herein, although AMP is readily converted to ATP for use in a luciferase-based assay, other nucleotide monophosphates (e.g., rCMP, rGMP and rUMP) can also be converted to ATP and used in such assays. Thus, in some cased the nucleic acid probes of the assay comprise other nucleosides and, in some cases, may contain little of no adenosine.

In some embodiments the assay uses a signal production system that consumes a molecule other than ATP (e.g., a nucleoside monophosphate, or another nucleoside triphosphate).

Simultaneous detection of multiple different target sequences (multiplexing) can be carried out in a number of ways. In one approach pools of guide RNAs that can “rule in/rule out” presence of a particular class of sequences are used as an initial screen (triage) followed by a second assay. For example, a first test could use gRNA that targets all beta lactamase genes. If the first test is positive further test(s) are carried out to identify which specific beta lactamase gene variant(s) is present. In another approach, endonuclease (e.g., Cas13a) variants with different specificities for cis cleavage targets (see Gootenberg et al, 2018, “Multiplexed and portable nucleic acid detection platform with Cas13, Cas12a, and Csm6” Science 360:439-444) can be used.

### IV. Detection

The monoribonucleotides generated by the exonuclease activity may be quantitated by any suitable method. By detecting and optionally quantitating the monoribonucleotides (or determining whether they exceed a particular threshold), the user can determine whether a nucleic acid sample contains a target sequence based on the quantity of monoribonucleotides generated.

One such method comprises converting the monoribonucleotides to adenosine triphosphate (ATP), and measuring the ATP by an appropriate method. ATP can be measured by HPLC, or using molecular probes that are specific for ATP (see, e.g. Rajendran et al., 2016, Biol. Bull. 2331:73-84). Alternatively, the ATP generated may be coupled to readouts such as a pH change, a visible spectrum dye color change, an electrical current, a temperature change, or bioluminescence. For example, The ATP Assay Kit from Abcam relies on the phosphorylation of glycerol to generate a product that is easily quantified by colorimetric or fluorometric methods.

In one approach, an assay with a bioluminescence readout is used. A suitable bioluminescent ATP detection system is based on luciferase. The term “luciferase” as used in this disclosure refers broadly to a class of oxidative enzymes that produce bioluminescence. A variety of organisms regulate their light production using different luciferases in a variety of light-emitting reactions. Suitable luciferases are native to animal species including fireflies, marine animals such as copepods, jellyfish, and the sea pansy, luminous fungi, like the Jack-O-Lantern mushroom, as well as examples in other kingdoms including luminous bacteria, and dinoflagellates.

Firefly luciferase generates light from luciferin in a multistep process. First, D-luciferin is adenylated by MgATP to form luciferyl adenylate and pyrophosphate. After activation by ATP, luciferyl adenylate is oxidized by molecular oxygen to form a dioxetanone ring. A decarboxylation reaction forms an excited state of oxyluciferin, which tautomerizes between the keto-enol form. The reaction finally emits light as oxyluciferin returns to the ground state. T. O. Baldwin, Structure. 4(3):223-28, 1996. Bacterial luciferase is encoded by the luxCDABE operon that is usually sourced from Photorhabdus luminescens, Vibrio harveyi or Vibrio fischeri. Bacterial luciferase catalyzes the oxidation of reduced flavin mononucleotide (FMNH2) and myristyl aldehyde to myristic acid and FMN, a reaction that liberates light at 490 nm. The luxAB genes encode the heterodimeric luciferase, while the luxCDE genes encode the enzymes required for generation of the myristyl aldehyde substrate from myristol ACP. A consideration when choosing the type of luciferase to use is that the firefly enzyme requires exogenous addition of the decanal substrate, whereas the bacterial system can be engineered (by inclusion of luxCDE in the operon) to generate the substrate endogenously. K. M. Devine, Methods in Microbiology, 2012. Both native and genetically engineered luciferase proteins are available commercially.

Optionally, nucleoside triphosphates already present in the reaction mixture (for example, from the starting sample) can be removed and then nucleoside monophosphates generated by the exonuclease are converted to nucleoside triphosphates and quantitated. For example, the sample is treated with a first reagent containing adenylate cyclase, pyrophosphatase PAP, and polyphosphate; and then with a second reagent containing adenylate kinase, luciferin, and luciferase: US 2013/0109037 A1.

Commercially available luciferase-based kits may be used to quantitate ATP. For example, the Readiuse™ Rapid Luminometric ATP Assay Kit, the ATP Determination Kit from Thermofisher, ENLITEN® ATP Assay System from Promega, the ATP Bioluminescence Assay Kit from Sigma-Aldrich. The AMPGlo® Kit from Promega is already set up to quantitate adenosine mononucleotide, converting AMP to ATP. Two reagents are supplied: one to terminate the AMP-generating enzymatic reaction, remove ATP and convert AMP produced into ADP, and a second reagent to convert ADP to ATP, which is used to generate a luminescence in a luciferase reaction.

### UMP and CMP Assays

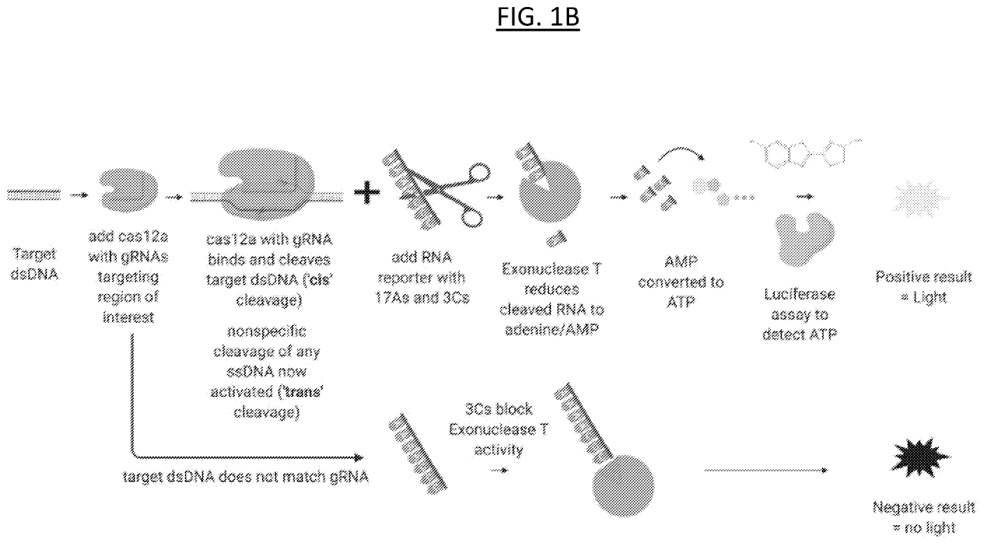

As an alternative to an ATP based assay (e.g., an ATP-based luciferase assay), the polyadenosine reporter oligonucleotide may be substituted with an rU, rG or rC polymer, for example, quantitating the results with Promega's UMP/CMP-Glo™ glycosyltransferase assay kit. FIG. 2 shows the chemistry underlying the UMP/CMP-Glo kit. This is a homogeneous, single-reagent-addition method to detect UMP or CMP formation. After a glycosyltransferase reaction, UMP/CMP Detection Reagent is added to simultaneously convert the UMP or CMP product to ATP and generate light in a luciferase reaction.

### V. Illustration

FIG. 1B depicts a particular implementation of the NANCI method. In Step 1, Cas12a is complexed with a guide RNA targeting a sequence of interest and added to a sample. An oligonucleotide probe designated “rA17rC3” is added that has the following structure, where rA indicates an adenosine ribonucleotide and rC indicates a cytidine ribonucleotide.

[SEQ ID NO: 4]
5′-rArArArArArArArArArArArArArArArArArCrCrC-3′

This probe is designed to work with RNAse T, and contains the following features:
1. An unblocked 5′ end because RNAse T digests only in the 3′ to 5′ direction;
2. Seventeen RNA adenosine bases which will ultimately be digested into AMP molecules by RNAse T.
3. Three RNA cytdine bases on the 3′ end which act as a block to RNAse T.
   The Cas12a, guide RNA, sample, and probe mixture is incubated at 37° C.

In Step 2, RNAse T (also called Exonuclease T) is added to the mixture and incubated at 25° C. for 1 h. This enzyme will degrade the 3′ ends of any Cas12a-cleaved probe molecules to generate AMP monomers.

In Step 3, to detect AMP monomers, the entire sample is used as a substrate for generation and detection of bioluminescence. Suitability can be established using Promega's AMP-Glo® assay. This kit converts AMP to a light signal using a two-step process described in pre-grant patent publication US 2013/0109037. Any preexisting ATP is removed while AMP is converted to ADP. ADP is then converted to ATP, which generates photons through the luciferase system. This is read in a standard plate reader in luminescence mode. The kit employs a slow-acting luciferase, so the light signal can be read over the course of several hours, and the signal integrated over that time to increase sensitivity.

In other implantations of the assay method, other alternative Cas12 variants with different activity levels and different specificities may be used. Fuchs et al., bioRxiv (2019) doi:10.1101/600890. Another implementation of the assay uses Cas13a6, which targets RNA instead of DNA, or Cas147 which targets single stranded DNA: both of these enzymes also have the trans-cleavage activation property.

### VI. Samples and Sample Preparation

As already described, the assay systems of this disclosure may be used to test for a particular nucleic acid sequences from any source suspected of containing nucleic acids. The source being tested may be prepared in a suitable fashion to constitute the “sample” referred to in the claims below.

The nucleic acids in the sample may be DNA and/or RNA from a human or a non-human animal and nucleic acids from a prokaryote or population of prokaryotes. The nucleic acid being tested may be genomic DNA, mitochondrial DNA, mRNA, rRNA, or cDNA. The nucleic acid being tested may be from a microorganism or population of microorganisms. The microorganisms may be pathogenic to humans or other animals. The nucleic acid being tested may be collected from a fluid or tissue obtained for clinical assessment.

In some embodiments, nucleic acids may be collected from a biological, clinical, agricultural, environmental or other source, and may be amplified to produce amplification products (amplicons) for use in the NANCI assay disclosed herein. Suitable amplification methods are well known and include without limitation multiple displacement amplification (MDA), polymerase chain reaction (PCR), and transcription mediated amplification (TMA).

### VII. Kits and their Use

This disclosure provides reagents and reagent combinations for carrying out assay methods and/or sample preraparation as already described. The reagents are provided in suitable container(s), optionally accompanied by or sold in conjunction with reaction vessels, and written information about the use of the reagents or reagent combinations in performing one or more of the assays described herein.

Included are assay kits for determining whether a sample contains a particular nucleic acid sequence, small molecule, or protein of interest. Such kits may include an enzyme that generates monoribonucleotides if the target of interest is present: for example, a CRISPR associated protein (Cas) that has endonuclease collateral activity, a guide RNA that is complementary to the target sequence, and a reporter oligonucleotide. The Cas and the guide RNA form a complex with nucleic acid in the sample if the nucleic acid contains the target sequence, thereby causing the collateral activity of the Cas to cleave the reporter oligonucleotide internally.

The kit may also include a means for generating monoribonucleotides from reporter oligonucleotide molecules that have been cleaved. The means for generating monoribonucleotides from reporter nucleotide molecules that have been cleaved is an RNAse T, and the reporter oligonucleotide has been blocked at the 3′ end to prevent it from being a substrate for the RNAse T unless it is cleaved internally by the collateral activity of the Cas. The kit may also contain a means for quantitating monoribonucleotides, exemplified by a means for generating bioluminescence in response to monoribonucleotides, exemplified by a luciferase and one or more needed substrates, such as a luciferin. The reagents may all be provided separately or premixed.

The methodology and products of the invention may also be adapted as a biosensing platform for sensitive detection of diverse small molecules. Liang et al., Nature Communications, DOI:10.1038/s41467-019-11648-1, 2019. The methodology and products of this invention may also be adapted for ultrasensitive detection of proteins, with target recognition achieved through proximity binding. Yongya et al., bioxRiv doi: 10.1101/734582, 2019. After the monoribonucleotides have been quantitated as described above, mutatis mutandis, the user determines whether a sample contains a particular small molecule or protein of interest.

### VIII. Glossary

The terms “collateral activity” and “trans activity” used in this disclosure refer to nuclease activity of the sequence-recognizing complex (typically endonuclease activity) that cleaves nucleic acid that is not proximal to the binding site of the complex (i.e., the sequence complementary to the gRNA). Typically, the collateral activity is not sequence specific. The assay can be set up with a reporter oligonucleotide that is also present in the reagent mixture as a source of the monoribonucleotides.

In the context of the claimed invention, a “reporter oligonucleotide” or a “nucleic acid reporter probe” is an oligonucleotide of any length that is used as a reagent in a reaction mixture, in combination with a Cas protein and a sample which may or may not contain a target polynucleotide sequence.

As used herein rA indicates an adenosine ribonucleotide, rC indicates a cytidine ribonucleotide, rU indicates a uridine ribonucleotide, rT indicates a thymidine (5-methyluridine) ribonucleotide, rG indicates a guanosine ribonucleotide; dA indicates an adenosine deoxyribonucleotide, dC indicates a cytidine deoxyribonucleotide, dT indicates a thymidine deoxyribonucleotide, dG indicates a guanosine deoxyribonucleotide

As used herein an “oligonucleotide” is a polymer of nucleotides without reference to a particular length. Generally an oligonucleotide has a length of 10 to 1000 nucleotides, often 20-500 nucleotides, and sometimes 20-200 nucleotides. An oligonucleotide may be modified at its termini or internally. An oligonucleotide may comprise nucleotides selected from rA, rT, rU, rG, rC, be modified at its termini or internally

An oligonucleotide is “blocked” if it contains a nucleotide, modification, or structural feature that prevents digestion by a 5′ or 3′ exonuclease.

The terms “nucleotide monomer,” “NMP,” “nucleoside monophosphate,” “nucleotide monophosphate,” and (as will be clear from context) “nucleotide” are used interchangeably. Nucleotides comprise a nitrogenous base (adenine, thymine, cytidine, guanine), a sugar (ribose, deoxyribose), and a phosphate group, or analogs of any of the components. Exemplary ribonucleosides are cytidine, uridine, adenosine, guanosine, thymidine and the corresponding nucleoside monophosphates are adenosine monophosphate (AMP), cytidine monophosphate (CMP), thymidine monophosphate (TMP), guanosine monophosphate (GMP), and uridine monophosphate (UMP), and their cognate deoxyribonucleoside monophosphates (dAMP, dCMP, dTMP, dGMP, dUMP.)

### IX. Additional References

The following references are referred to in various places of the text above. Other pertinent publications are cited and annotated elsewhere in this disclosure.
1. Chen, J. S. et al. CRISPR-Cas12a target binding unleashes indiscriminate single-stranded DNase activity. Science 360, 436 (2018).
2. Fuchs, R. T., Curcuru, J., Mabuchi, M., Yourik, P. & Robb, G. B. Cas12a trans-cleavage can be modulated in vitro and is active on ssDNA, dsDNA, and RNA. bioRxiv (2019) doi:10.1101/600890.
3. Li, J., Macdonald, J. & von Stetten, F. Review: a comprehensive summary of a decade development of the recombinase polymerase amplification. The Analyst 144, 31-67 (2019).
4. Zuo, Y. & Deutscher, M. P. The Physiological Role of RNAse T Can Be Explained by Its Unusual Substrate Specificity. J. Biol. Chem. 277, 29654-29661 (2002).
5. Goueli, S. A., Hsiao, K. & Zegzouti, H. Methods for detecting adenosine monophosphate in biological samples. (2013).
6. Gootenberg, J. S. et al. Nucleic acid detection with CRISPR-Cas13a/C2c2. Science 356, 438-442 (2017).
7. Harrington, L. B. et al. Programmed DNA destruction by miniature CRISPR-Cas14 enzymes. Science 362, 839-842 (2018).
8. Viswanathan, M., Dower, K. W. & Lovett, S. T. Identification of a potent DNAse activity associated with RNAse T of Escherichia coli. J. Biol. Chem. 273, 35126-35131 (1998).

### X. Examples

### Example 1: Reagents

Materials:
EnGen® Lba Cas12a (Cpf1) from New England Biolabs. EnGen Lba Cas12a (Cpf1) is a programmable DNA endonuclease guided by a single guide RNA (gRNA). Targeting requires a gRNA complementary to the target site as well as a 5′ TTTV protospacer adjacent motif (PAM) on the DNA strand opposite the target sequence. Cleavage by EnGen Lba Cas12a (Cpf1) occurs ˜17 bases 3′ of the PAM and leaves 5′ overhanging ends.
gRNA: synthetic from Integrated DNA Technologies (IDT)
target dsDNA
RNAse T in NEB4 buffer from New England Biolabs. RNAse T is also known as Exonuclease T. Single-stranded (ssDNA or RNA) specific exonuclease. Catalyzes the removal of nucleotides from linear single-stranded DNA or RNA in the 3′ to 5′ direction.

Promega AMPGlo® Assay. The AMP-Glo Assay is a homogeneous assay that generates a luminescent signal from any biochemical reaction that produces AMP. It can be used to measure the activity of a broad range of enzymes, such as cyclic AMP-specific phosphodiesterases, aminoacyl-tRNA synthetases, DNA ligases and ubiquitin ligases or enzymes modulated by AMP.

The assay quantitatively monitors AMP concentration in a biochemical reaction in a wide range of plates, including high-throughput formats. The stable luminescent signal eliminates the need for an injector-equipped luminometer and allows batch-mode processing of multiple plates. The assay can be used to determine the AMP produced either in the presence or absence of ATP as a substrate.

Two reagents are supplied: one to terminate the AMP-generating enzymatic reaction, remove ATP and convert AMP produced into ADP, and a second reagent to convert ADP to ATP, which is used to generate a luminescence in a luciferase reaction. The AMP-Glo™ Assay is well suited for monitoring AMP produced in biochemical reactions catalyzed by enzymes that do not use ATP as a substrate, such as cAMP-dependent phosphodiesterases (PDE) and bacterial DNA ligases.

### Example 2: Methodology

A procedure for performing an exemplary assay is as follows:

A) Preassembly of Cas12a with gRNA and a dsDNA containing a target sequence
1. Dilute synthetic gRNA to 35.2 ng/μL. For 11° C. at 100 μM, Add 5 μL crRNA at 100 μM to 180.5 μL of water to get 35.2 ng/μL
2. Dilute Cas12a to 1 μM in 1×NEB 2.1 buffer
3. Prepare a MM (master mix) of Cas12a, buffer, water and gRNA, in that order. For no Cas12a control, add 1×NEB 2.1 buffer. For no gRNA control, add water. If using different gRNA, leave gRNA out of MM and add 0.75 μL gRNA to each tube. Mix via pipette, tapping or a VERY gentle quick vortex.
4. Add 4 μL of MM to each PCR strip tube
5. Add 1 μL of target at appropriate concentration. Mix via pipette or tapping. Spin down tubes before incubation

Preassembly

| | Stock | Amount per | Final | |
|---|---|---|---|---|
| | Concentration | Reaction (1X) | Concentration | Reagent |
| 1 | 1 μM | 0.5 μL | 100 nM | Cas12a |
| 2 | 10 X | 0.5 μL | 1X | 10 X NEB 2.1 |
| | | | | Buffer |
| 3 | | 2.25 μL | | water |
| 4 | 2.66 μM | 0.75 μL | 400 nM | crRNA |
| | (35.2 ng/μL) | | | |
| 5 | 500 nM | 1 μL | 100 nM | target dsDNA |
| 6 | | 5 μL | (total reaction | |
| | | | volume) | |

6. Incubate at 37° C. for 30 min (heated lid off or at 40° C.) and then cool to 4° C. Can be left at 4° C. for up to 4 h.

(B) Reporter Addition
7. Thaw 100 μM aliquots of reporter (i.e., r17Ar3C, r17A-AAAAA-phos, r17A-GGAGG-ddC, etc.)
8. Dilute to 16 μM by taking 10 μL and adding to 52.5 μL of water
9. Prepare MM (master mix) of reporter, buffer and water. Vortex to mix. Dispense to PCR strip tubes for easy transfer via multichannel
10. Add 5 μL of MM to 5 μL of Cas12a reaction. Mix via pipette or light tapping. Spin down tubes before incubation

Reporter Reaction

| | Stock | Amount per | Final | Reagent |
|---|---|---|---|---|
| | Concentration | Reaction 1X) | Concentration | |
| 1 | 16 μM | 2.5 μL | 4 μM | reporter |
| 2 | 10 X | 0.5 μL | IX | NEB 2.1 Buffer |
| 3 | | 2 μL | | water |
| 4 | 100 nM: 400 nM: | 5 μL | 50 nM: 200 nM: | Cas12a:crRNA: |
| | 100 nM | | 50 nM | target DNA |
| 5 | | 10 μL | (total reaction | |
| | | | volume) | |

11. Incubate at 37° C. for 3 h (heated lid off or at 40° C.)

(C) RNAse T Digestion
12. Prepare ExoT MM (RNAse T master mix). Keep ExoT on ice. Thaw buffer 20 min before end of Reporter reaction

RNAse T Reaction

| | | Amount per |
|---|---|---|
| | Reagent | Reaction (IX) |
| 1 | Cas12a-reporter | 3 μL |
| | reaction mixture | |
| 2 | 10 X NEB buffer 4 | 0.6 μL |
| 3 | water | 1.9 μL |
| 4 | RNAse T | 0.5 μL |
| 5 | Total volume | 6 μL |

13. Add 3 μL of Exo T MM to 3 μL of Cas12a reaction. Mix via pipette or light tapping. Spin down tubes before incubation
14. Incubate at 25 C for 1 h and heat inactivate at 65° C. for 20 min with heated lid set to 105° C. (RNAse protocol on Spec Ops™ PCR machine)

(D) Use of AMPGIo® Kit to Detect AMP
15. Thaw AMP GLO Reagent 1 when you put Exo T incubation in, to give it an hour to come to room temperature
16. Add 5 μL of AMP GLO Reagent 1 to black low volume 384 plate using electronic single channel pipette. Dispense the first and last aliquot into the tube—these volumes are less accurate. May need to do the last few by hand. Skip every other column so that you can transfer via multichannel.
17. Transfer 5 μL of Cas12a-ExoT reaction to Reagent 1 in black 384 plate. Mix via pipette 5 times and spin down plate.
18. Incubate at room temperature on bench for 1 h
19. Thaw Kinase GLO Reagent (in single use aliquots) when incubation with Reagent 1 starts, to give it an hour to come to room temperature. Leave Reagent II in freezer or on ice until use.
20. Just before end of Reagent 1 incubation, prepare AMP Detection buffer—10 μL of Reagent II to 1 mL of Kinase GLO Reagent. Dispense to PCR strip tubes for multichannel transfer
21. Add 10 μL AMP Detection buffer to each well using multichannel. Mix via pipette 5 times and spin down plate.
22. Place on the plate reader and let incubate at room temperature for an hour while reading plate. Use white low volume 384 well plate.

(E) Read Luminescence, Top Read. Standard Kinetic Reading Settings: 1 h, 10 Min Readings, 500 Msec Integration.

### Example 3: Implementation and Data Obtained Thereby

A synthetic Cas12a guide RNA ordered from IDT targeting the fbiC gene from Mycobacterium tuberculosis was prepared and complexed with Cas12a. A synthetic double stranded DNA molecule containing the target sequence and an appropriately positioned PAM site was added at a concentration of 25 nM.

The probe rA17rC3 was also added and the mixture was incubated for 3 h at 37° C. RNAse T was added and the mixture was further incubated for 1 h at 25° C., and then heat inactivated at 65° C. for 20 min. The entire mixture was used as a substrate in the AMP-Glo® Assay kit (Promega Corp.), following the manufacturer's instructions, and the sample, along with the various controls indicated, was read on a SpectraMax™ M3 plate reader (Molecular Devices) in luminescence mode with an integration time of 0.5 seconds. Relative luminescence units were converted to AMP concentration in μM using an AMP standard curve as prescribed by the AMP-Glo® Assay kit.

FIG. 2 provides data from this assay. In this implementation the NANCI assay detected the target DNA at a concentration of 25 nM.

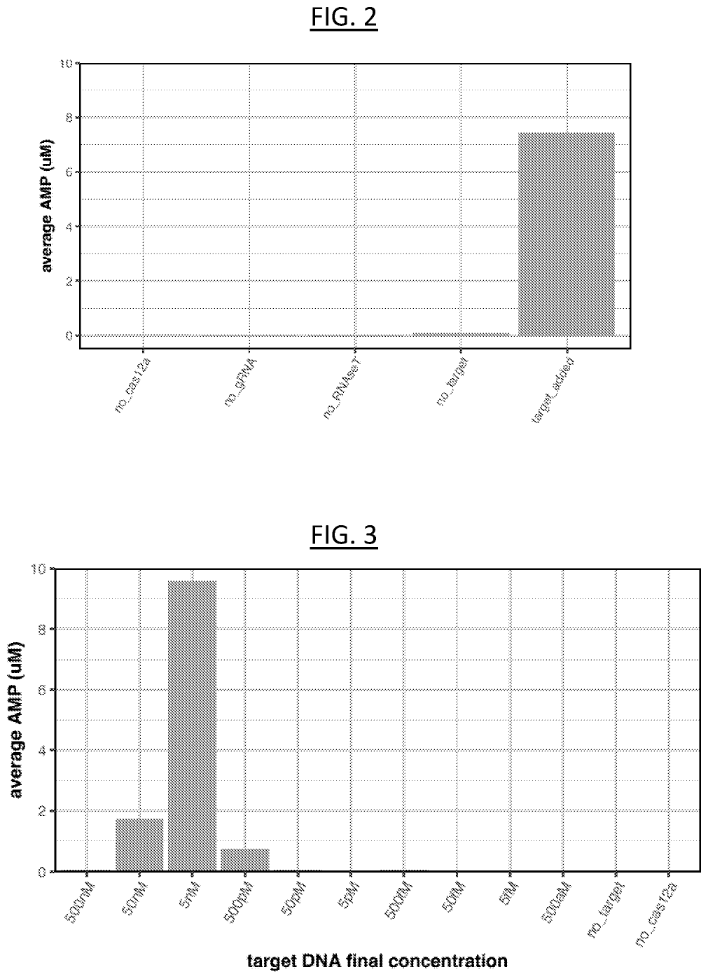

FIG. 3 shows results of another experiment. The procedure above was repeated with varying concentrations of target DNA. A concentration of 500 pM DNA is clearly detectable with the NANCI assay.

The invention has been described in this disclosure with reference to the specific examples and illustrations. The features of these examples and illustrations do not limit the practice of the claimed invention, unless explicitly stated or otherwise required. Changes can be made and equivalents can be substituted to adapt to a particular context or intended use as a matter of routine development and optimization and within the purview of one of ordinary skill in the art, thereby achieving benefits of the invention without departing from the scope of what is claimed and their equivalents.

For all purposes in the United States of America, each and every publication and patent document referred to in this disclosure is incorporated herein by reference in its entirety to the same extent as if each such publication or document was specifically and individually indicated to be incorporated herein by reference.

1. An assay method for determining whether a sample contains a target polynucleotide comprising a target nucleic acid sequence, the method comprising:
(a) combining the sample with a CRISPR associated (Cas) protein that has endonuclease collateral activity, a guide RNA (gRNA), and a reporter oligonucleotide, to form a mixture,
wherein, if the target polynucleotide comprising the target nucleic acid sequence is present in the sample, the Cas protein and the gRNA cause the collateral activity of the Cas protein to cleave the reporter oligonucleotide, thereby producing a cleaved reporter oligonucleotide;
(b) generating monoribonucleotides from the cleaved reporter oligonucleotide produced in step (a);
(c) determining whether monoribonucleotides have been generated in step (b); and
(d) characterizing the sample as containing the target polynucleotide with the target nucleic acid sequence if monoribonucleotides are detected in step (c).

2. The method of claim 1, wherein monoribonucleotides are produced from the cleaved reporter oligonucleotide by an exonuclease.

3. The method of claim 2 wherein monoribonucleotides are generated from the reporter oligonucleotide in step (b) by an exonuclease only if the reporter oligonucleotide has been cleaved by the Cas protein.

4. The method of claim 2, wherein the monoribonucleotides are generated from the reporter oligonucleotide by including RNAse T in the mixture in step (a) or adding an RNAse T to the mixture in step (b).

5. The method of claim 1 wherein the reporter oligonucleotide is a ribonucleotide polymer (RNA).

6. (canceled)

7. The method of claim 6 wherein the RNA comprises at least 10 consecutive adenosines.

8. The method of claim 1 wherein the reporter is an RNA-DNA chimera.

9. The method of claim 1, comprising removing at least one residual nucleotide triphosphate from the mixture before step (c), then converting the monoribonucleotides produced in Step (b) to adenosine triphosphate, and quantitating the adenosine triphosphate produced by the conversion.

10. The method of claim 1 wherein the target polynucleotide is double stranded DNA or single stranded RNA.

11-12. (canceled)

13. The method of claim 1, wherein the monoribonucleotides are detected in step (c) by bioluminescence.

14. The method of claim 1, wherein the monoribonucleotides are quantitated in step (c) by including or adding a luciferase in the mixture, and measuring bioluminescence produced thereby.

15. The method of claim 1, wherein in step (a) a gRNA-Cas protein complex is formed prior to adding the reporter oligonucleotide.

16. (canceled)

17. The method of claim 13 wherein the microorganism is a human pathogen.

18. An assay kit for determining whether a nucleic acid sample contains a target polynucleotide, said target polynucleotide comprising a target nucleic acid sequence, the kit comprising:
(1) a reporter oligonucleotide;
(2) a means for cleaving the reporter oligonucleotide to produce a cleaved reporter oligonucleotide, wherein the means comprise a CRISPR associated (Cas) protein that has endonuclease collateral activity and a guide RNA (gRNA) that targets the Cas protein to the target nucleic acid sequence;
(3) a means for generating nucleoside monophosphate from the cleaved reporter oligonucleotide, wherein the means comprises at least one exonuclease.

19. The kit of claim 18, further comprising a means for measuring monoribonucleotides generated from the cleaved reporter oligonucleotide.

20. The kit of claim 18, wherein the reporter oligonucleotide is blocked at least one terminus so that the reporter oligonucleotide is not a substrate for the exonuclease(s).

21. The kit of claim 18, wherein the reporter oligonucleotide is at least 50 nucleotides in length and at least 60% of nucleotide monomers in the reporter oligonucleotide are adenosine.

22. The kit of claim 18, wherein the Cas protein is a Cas12a protein, a Cas13a protein, or a Cas14 protein.

23. The kit of claim 22, wherein the means for measuring monoribonucleotides comprises a luciferase and a luciferin.

24. The kit of claim 18, wherein the exonuclease is an RNAse T, and the reporter oligonucleotide comprises a polyadenosine sequence blocked at its 3′ terminus, such that the reporter oligonucleotide is not a substrate for the RNAse T.


SEQUENCE LISTING (4 sequences)

SEQ ID NO: 1 - DNA, 8 bp, Homo sapiens
aaaatttg

SEQ ID NO: 2 - RNA, 8 nt, Homo sapiens
aaaaaccc

SEQ ID NO: 3 - DNA, 20 bp, Homo sapiens
aaaaaaaaaaaaaaaaaaaa

SEQ ID NO: 4 - RNA, 20 nt, Homo sapiens
aaaaaaaaaaaaaaaaaccc